(19) United States
(12) Patent Application Publication
(10) Pub. No.: US 2026/0115216 A1
(43) Pub. Date: Apr. 30, 2026

(54) NUCLEIC ACID PRECURSOR

(71) Applicant: KYUSHU UNIVERSITY, NATIONAL UNIVERSITY CORPORATION, Fukuoka (JP)

(72) Inventors: Takahisa ANADA, Fukuoka-shi, Fukuoka (JP); Masaru TANAKA, Fukuoka-shi, Fukuoka (JP); Shingo KOBAYASHI, Fukuoka-shi, Fukuoka (JP); Michiharu KAWAHARA, Fukuoka-shi, Fukuoka (JP)

(73) Assignee: KYUSHU UNIVERSITY, NATIONAL UNIVERSITY CORPORATION, Fukuoka (JP)

(21) Appl. No.: 19/102,048

(22) PCT Filed: Aug. 9, 2023
(86) PCT No.: PCT/JP2023/029131

§ 371 Date: Nov. 18, 2025

(30) Foreign Application Priority Data
Aug. 10, 2022 (JP) 2022-128175

(51) Int. Cl. A61K 31/7076; A61P 3/00; C07H 19/207

(52) U.S. Cl. CPC A61K 31/7076; A61P 3/00; C07H 19/207

(57) ABSTRACT

The present invention provides compounds for treating or preventing a state of decreased intracellular ATP levels.

The present invention relates to a compound represented by formula (I):

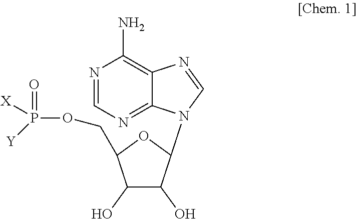

in which
X and Y are the same or different and each represents a substituent selected from Substituent group A: This is the case when the atom connected to the phosphorus atom is oxygen; a group represented by formula 1: —O—R1 in which R1 is selected from the group consisting of a hydrogen, a C1 to C8 alkyl group, a C1 to C3 alkylaryl group, CH₃OCH₂—, CH₃OCH₂CH₂—, an ester group, a phenyl group, a pyridyl group, a benzyl group, an indolyl group, and a naphthyl group, wherein the aromatic ring or heterocyclic ring thereof may be substituted with one to three of the following functional groups;
or a substituent selected from Substituent groups B: This is the case when the atom connected to the phosphorus atom is nitrogen; a group represented by formula 2: —R2—R3 in which R2 is a residue collectively referred to as an aminocarboxylic acid, and R3 is a substituent that binds to the carboxylic acid part of the aminocarboxylic acid in R2; or a pharmaceutically acceptable salt or solvate thereof, and a pharmaceutical composition which comprises the same.

### TECHNICAL FIELD

The present application claims the priority under the Paris Convention with respect to the Japanese Patent Application No. 2022-128175 filed on Aug. 10, 2022, which is incorporated herein by reference in its entirety.

The present invention generally relates to nucleic acid precursors and pharmaceutical compositions comprising the same, and specifically to prodrug forms of ATP and pharmaceutical compositions containing the same.

### BACKGROUND TECHNOLOGY

All living organisms, from unicellular organisms to humans, live by performing metabolism within their cells. The energy source is adenosine triphosphate (ATP), which is utilized as intracellular energy currency universally by all organisms. Recently, ATP has attracted attention not only for its role as the intracellular energy currency but also for its ability to dissolve proteins at high concentrations within the cell and to dissolve droplets formed within the cell (Non-Patent Document 1).

ATP can be administered intravenously and used as a medication for conditions such as pulmonary hypertension, blood pressure during anesthesia and surgery, lung cancer, multiple organ failure, cancer-related weight loss, acute renal failure, cystic fibrosis, ischemia, and cardiac stress testing. Further, the deficiency of ATP is one of the main causes of delayed healing of diabetic chronic wounds (Non-Patent Document 2). Aging leads to a decline in the energy metabolism function of cells, and causes senescence, which deteriorates physical functions, reporting that there is a correlation between the decline in energy metabolism due to aging and the decrease in intracellular ATP levels. This means that aging decreases the intracellular ATP levels and the cell's stress resistance, thereby triggering various age-related diseases.

Adenosine derivatives such as ATP have an extremely low stability in blood with a half-life in blood of about 10 seconds, and the administration as they are is not efficient. Additionally, ATP has the property of hardly entering the cell from the extracellular application, since ATP has no cell membrane permeability due to the negative charge.

### CITATION LIST

### Non-Patent Documents

Non-Patent Document 1: A. Patel et al., Science, 356, 753-756 (2017)
Non-Patent Document 2: J. Drug Targeting, 2015, 23, 580-596

### SUMMARY OF THE INVENTION

### Problems to be Solved by the Invention

Since ATP has a low cell membrane permeability, the addition of ATP to cells as it is hardly exerts any effect as ATP. Therefore, the introduction techniques of ATP into cells comprising encapsulating ATP in lipid bilayers (liposomes) or nanoparticles have been reported as ATP delivery methods at the present time. However, those techniques are limited in utility, and do not reach a level where ATP can be used as a medicine, meaning that there is currently very little data indicating that the intracellular ATP concentration could be increased.

### Means to Solve Problems

In this situation, the present inventors have earnestly studied aiming at increasing the level of ATP within the cells.

According to the present invention, prodrug forms of ATP, such as phosphoramidate AMPs (ATP prodrugs), are capable of penetrating the cell membrane due to its high hydrophobicity, unlike ATP which cannot pass through the cell membrane. The present inventors discovered that this compound was a prodrug that exerts an effect of increasing the intracellular ATP levels by converting AMP to ATP upon undergoing intracellular metabolism, and thus have completed the present invention.

Specifically, the present invention encompasses the aspects as follows.

### Compounds

[1]

A compound represented by formula (I):

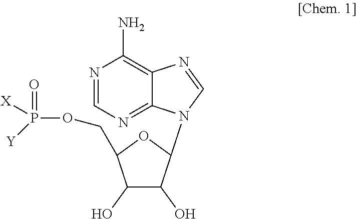

in which
X and Y are the same or different and each represents a substituent selected from Substituent group A represented below, or a substituent selected from Substituent groups B and C represented below, provided that X and Y are not simultaneously any substituent selected from Substituent group A;
Substituent group A: This is the case when the atom connected to the phosphorus atom is oxygen; a group represented by formula 1:

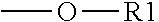

in which R1 is selected from the group consisting of a hydrogen, a C1 to C8 alkyl group, a C1 to C3 alkylaryl group, CH₃OCH₂—, CH₃OCH₂CH₂—, an ester group, a phenyl group, a pyridyl group, a benzyl group, an indolyl group, and a naphthyl group, wherein the aromatic ring or heterocyclic ring thereof may be substituted with one to three of the following functional groups: halogen, C1-C6 alkyl, C2-C6 alkenyl, C1-C6 halogenated alkyl, C1-C6 alkoxy, C1-C6 halogenated alkoxy, phenyl, hydroxy C1-C6 alkyl, C3-C6 cycloalkyl, C1-C6 alkyl carbonyl, C3-C6 cycloalkyl carbonyl, carboxy Ce—Ce alkyl, hydroxyl group, amino group, cyano group, nitro group, trimethylsilyl group;
Substituent group B: This is the case when the atom connected to the phosphorus atom is nitrogen; a group represented by formula 2:

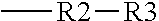

in which R2 is a residue collectively referred to as an aminocarboxylic acid;
R3 is a straight-chain C1 to C6 alkyl group or a branched or cyclic C3 to C6 alkyl group that binds to the carboxylic acid part of the aminocarboxylic acid in R2, preferably, for example, methyl, ethyl, n-propyl, isopropyl, cyclopropyl, n-butyl, isobutyl, sec-butyl, tert-butyl, n-pentyl, isopentyl, sec-pentyl, tert-pentyl, neopentyl, 1-methylpentyl, n-hexyl, isohexyl, sec-hexyl, tert-hexyl, neohexyl, and the like; a C4 to C8 cycloalkyl group that may be substituted with a C1 to C8 alkyl group; a tetrahydropyranyl group; a benzyl group that may be substituted with a halogen, a C1 to C8 alkyl group, or a C1 to C6 alkoxy group; a 2-phenylethyl group that may be substituted with a halogenated phenyl group; and an indole group; and
Substituent group C: This is the case when the atom connected to the phosphorus atom is nitrogen; C3 to C5 heterocyclic rings, including a morpholine ring, a piperazine ring, a thiomorpholine ring, or a cyclic structure represented by formula 3:

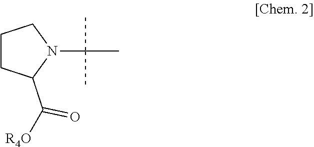

in which R4 is a hydrogen, a C1 to C8 alkyl group that may be substituted with a C3 to C6 cycloalkyl group: a C4 to C8 cycloalkyl group: a tetrahydropyranyl group: a benzyl group that may be substituted with a halogen, a C1 to C8 alkyl group, or a C1 to C6 alkoxy group: a 2-phenylethyl group that may be substituted with a halogenated phenyl group, or an indole group;
or a pharmaceutically acceptable salt or solvate thereof.
[2]

The compound according to [1], in which, in Substituent group A, a phenyl group that may be substituted with halogen, C1-C6 alkyl, or C1-C6 alkoxy;
or a pharmaceutically acceptable salt or solvate thereof.
   [3]

The compound according to [1], in which, in Substituent group B, the substituent is a group represented by formula 2:

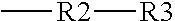

in which R2 is an amino acid residue selected from the group consisting of alanine, glycine, isoleucine, leucine, proline, methionine, phenylglycine, phenylalanine, valine, and asparagine;
   or a pharmaceutically acceptable salt or solvate thereof.
   [4]

The compound according to [1], in which, in Substituent group B, the substituent is a group represented by formula 2:

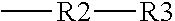

in which R3 is selected from the group consisting of methyl, ethyl, n-propyl, isopropyl, cyclopropyl, n-butyl, isobutyl, sec-butyl, tert-butyl, n-pentyl, isopentyl, sec-pentyl, tert-pentyl, neopentyl, 1-methylpentyl, n-hexyl, isohexyl, sec-hexyl, tert-hexyl, and neohexyl;
   or a pharmaceutically acceptable salt or solvate thereof.
   [5]

The compound according to [1], in which, in Substituent group B, the substituent is a group represented by formula 2:

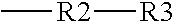

in which R3 is a straight-chain or branched C4 to C6 alkyl group;
   or a pharmaceutically acceptable salt or solvate thereof.
   [6]

The compound according to [1], in which the compound is represented by formula (II):

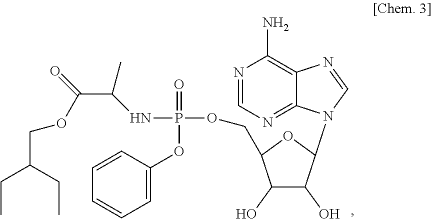 [SMILES: CCC(CC)COC(=O)C(C)NP(=O)(OCC1OC(n2cnc3c(N)ncnc32)C(O)C1O)Oc1ccccc1]

or
formula (III):

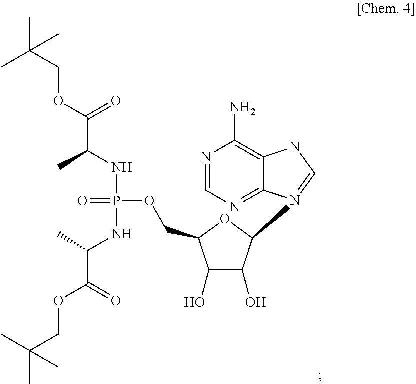

or a pharmaceutically acceptable salt or solvate thereof.

### Pharmaceutical Compositions

[7]

A pharmaceutical composition which comprises the compound according to any one of [1] to [6], or a pharmaceutically acceptable salt or solvate thereof.

[8]

The pharmaceutical composition according to [7], in which the composition is for treating or preventing a state of decreased intracellular ATP levels.

[9]

The pharmaceutical composition according to [8], in which the state of decreased intracellular ATP levels is associated with an aging state, as well as a non-alcoholic fatty liver disease and/or hepatitis.

[10]

The pharmaceutical composition according to [8], in which the state of decreased intracellular ATP levels is a mitochondrial disease, a cancer, or a hypoxic condition due to insufficient blood flows, or a tissue regeneration area accompanied by inflammation.

[11]

The pharmaceutical composition according to [8], in which the state of decreased intracellular ATP levels is a neurodegenerative disease.

[12]

The pharmaceutical composition according to [11], in which the neurodegenerative disease is selected from the group consisting of Parkinson's disease, Alzheimer's disease, and ALS.

### Effect of Invention

The phosphoramidate AMPs according to the present invention can penetrate the cell membrane due to their high hydrophobicity. These compounds exert their effect by converting from AMP to ATP on undergoing the intracellular metabolism, and consequently increase the intracellular ATP concentration.

### BRIEF DESCRIPTION OF DRAWINGS

FIG. 1 shows the chart of the NMR spectrum (400 MHz, ¹H NMR, MeOD) of(S)-2-ethylbutyl 2-(((S)-(((2R,3S4R,5R)-5-(6-amino-9H-purin-9-yl)-2,2-dimethyl-tetrahydrofur[3,4-d][1,3]dioxol-4-yl)methoxy)(phenoxy) phosphoryl)amino)propanoate as prepared in Example 1-1.

FIG. 2 shows the chart of the NMR spectrum (400 MHz, ¹H NMR, MeOD) of the phosphoramidate AMP (ATP prodrug) as prepared in Example 1.

FIG. 3 shows the chart of the FAB MS spectrum of the ATP prodrug.

FIG. 4 shows the chart of the NMR spectrum (400 MHz, ¹H NMR, MeOD) of the phosphoramidate AMP (ATP prodrug-neo) as prepared in Example 2.

FIG. 5 shows graphs illustrating the effects of the ATP prodrug on the intracellular ATP concentration. In the figure, “proATP” refers to the ATP prodrug.

FIG. 6 shows graphs illustrating the effects of adenosine, AMP, ATP, and the ATP prodrug on intracellular ATP concentration. In the figure, “proATP” refers to the ATP prodrug.

FIG. 7 shows a graph illustrating the effect of the ATP prodrug on the intracellular ATP concentration in normal cells. In the figure, “proATP” refers to the ATP prodrug.

FIG. 8 shows graphs illustrating the effects of increased ATP and stress load on aged cells. FIG. 8 (A) shows a graph illustrating the effects of the ATP prodrug on young cells and aged cells, whereas FIG. 8 (B) shows a graph illustrating the effect of the ATP prodrug on oxidative stress load affecting aged cells. The vertical axis of FIG. 8 (B) shows the percentage of cell numbers as 100% of the cell numbers in the absence of both hydrogen peroxide and proATP. In the figure, “proATP” refers to the ATP prodrug.

FIG. 9 shows a graph illustrating the examination of the cytotoxicity of the ATP prodrug as indicated by the release amount of LDH. In the figure, “proATP” refers to the ATP prodrug.

FIG. 10 shows graphs illustrating the intracellular ATP levels in two types of liver cells (cultured cells and primary cells) induced by the ATP prodrug. In the figure, “proATP” refers to the ATP prodrug.

FIG. 11 shows a graph illustrating the effect of the AMPK inhibitor on the intracellular ATP level induced by the ATP prodrug. In the figure, “proATP” refers to the ATP prodrug.

FIG. 12 shows a graph illustrating the effect of the ATP prodrug on the lifespan extension in nematodes. In the figure, “proATP” refers to the ATP prodrug.

FIG. 13 shows a graph illustrating the changes in the ATP levels within the nematodes induced by the ATP prodrug. In the figure, “proATP” refers to the ATP prodrug.

FIG. 14 shows a graph illustrating the effect of the ATP prodrug-neo on the intracellular ATP concentration.

FIG. 15 shows a graph illustrating the effect of the ATP prodrug-neo on the lifespan extension in nematodes. In the figure, “proATP” refers to the ATP prodrug.

### ASPECTS FOR CARRYING OUT INVENTION

Precursors of adenosine triphosphate (ATP), known as intracellular energy currency in all living organisms, have been invented. The compounds do not have any activity as ATP outside the cell, but, once they penetrate the cell membrane and enter the cell, they are converted into ATP through enzymatic reactions and hydrolysis, thereby functioning as energy currency.

### Compounds

In one aspect, the present invention relates to a compound represented by formula (I):

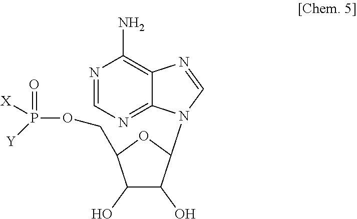

in which
X and Y are the same or different and each represents a substituent selected from Substituent group A represented below, or a substituent selected from Substituent groups B and C represented below, provided that X and Y are not simultaneously any substituent selected from Substituent group A;
Substituent group A: This is the case when the atom connected to the phosphorus atom is oxygen; a group represented by formula 1:

—O—R1
in which R1 is selected from the group consisting of a hydrogen, a C1 to C8 alkyl group, a C1 to C3 alkylaryl group, CH₃OCH₂—, CH₃OCH₂CH₂—, an ester group, a phenyl group, a pyridyl group, a benzyl group, an indolyl group, and a naphthyl group, wherein the aromatic ring or heterocyclic ring thereof may be substituted with one to three of the following functional groups: halogen, C1-C6 alkyl, C2-C6 alkenyl, C1-C6 halogenated alkyl, C1-C6 alkoxy, C1-C6 halogenated alkoxy, phenyl, hydroxy C1-C6 alkyl, C3-C6 cycloalkyl, C1-C6 alkyl carbonyl, C3-C6 cycloalkyl carbonyl, carboxy Ce—Ce alkyl, hydroxyl group, amino group, cyano group, nitro group, trimethylsilyl group;
Substituent group B: This is the case when the atom connected to the phosphorus atom is nitrogen; a group represented by formula 2:

—R2-R3
in which R2 is a residue collectively referred to as an aminocarboxylic acid;
R3 is a straight-chain C1 to C6 alkyl group or a branched or cyclic C3 to C6 alkyl group that binds to the carboxylic acid part of the aminocarboxylic acid in R2, preferably, for example, methyl, ethyl, n-propyl, isopropyl, cyclopropyl, n-butyl, isobutyl, sec-butyl, tert-butyl, n-pentyl, isopentyl, sec-pentyl, tert-pentyl, neopentyl, 1-methylpentyl, n-hexyl, isohexyl, sec-hexyl, tert-hexyl, neohexyl, and the like; a C4 to C8 cycloalkyl group that may be substituted with a C1 to C8 alkyl group; tetrahydropyranyl group; a benzyl group that may be substituted with a halogen, a C1 to C8 alkyl group, or a C1 to C6 alkoxy group; a 2-phenylethyl group that may be substituted with a halogenated phenyl group; and an indole group; and
Substituent group C: This is the case when the atom connected to the phosphorus atom is nitrogen; C3 to C5 heterocyclic rings, including a morpholine ring, a piperazine ring, a thiomorpholine ring, or a cyclic structure represented by formula 3:

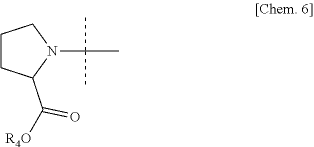

in which R4 is a hydrogen, a C1 to C8 alkyl group that may be substituted with a C3 to C6 cycloalkyl group: a C4 to C8 cycloalkyl group: a tetrahydropyranyl group: a benzyl group that may be substituted with a halogen, a C1 to C8 alkyl group, or a C1 to C6 alkoxy group: a 2-phenylethyl group that may be substituted with a halogenated phenyl group, or an indole group;
or a pharmaceutically acceptable salt or solvate thereof.

In formula (I), X and Y are the same or different and each represents a substituent selected from Substituent group A, Substituent group B, and Substituent group C, provided that X and Y are not simultaneously any substituent selected from Substituent group A. Therefore, the choices of substituent groups in X and Y are as follows:
X and Y=the substituents of Substituent group A and the substituents of Substituent group B
X and Y=the substituents of Substituent group A and the substituents of Substituent group C
X and Y=the substituents of Substituent group B and the substituents of Substituent group A
X and Y=the substituents of Substituent group B and the substituents of Substituent group B
X and Y=the substituents of Substituent group B and the substituents of Substituent group C
X and Y=the substituents of Substituent group C and the substituents of Substituent group A
X and Y=the substituents of Substituent group C and the substituents of Substituent group B
X and Y=the substituents of Substituent group C and the substituents of Substituent group C.

As used herein, the term “C1 to C8 alkyl group” refers to a straight or branched hydrocarbon chain that consists of the indicated number of carbon and hydrogen atoms, comprises no unsaturation, and is connected to the remainder of the molecule by single bonds. Examples include, but are not limited to, the methyl group, the ethyl group, the n-propyl group, the isopropyl group, the isobutyl group, the 3-methyl-1-pentyl group, the 4-methyl-1-pentyl group, the 3,3-dimethyl-1-butyl group, the t-butyl group, the pentyl group, the isopentyl group, and the hexyl group.

As used herein, the term “C1 to C3 alkylaryl group” refers to an aryl group that is substituted with an alkyl consisting of the indicated number of carbon and hydrogen atoms. The term “aryl” refers to the collective group of atoms remaining after the removal of one hydrogen atom from the aromatic ring of an aromatic hydrocarbon. In one embodiment of the present invention, examples of the aryl group include, but are not limited to, the phenyl group and the naphthyl group.

As used herein, “ester group” is the bond: —COO— formed by the dehydration condensation of a hydroxyl group and an acid.

As used herein, the term “C1-C6 alkoxy group” refers to a —O-alkyl group wherein the alkyl is defined as above. Examples of C1-C6 alkoxy groups include, but are not limited to, a lower alkyl alkoxy, a lower cycloalkyl alkoxy, and a lower bicycloalkoxy.

As used herein, the term “residues collectively referred to as amino carboxylic acids” is not particularly limited as long as it has a structure that contains an amino group and a carboxylic acid, and typically includes an amino acid residue.

As used herein, the term “amino acid residue” refers to a natural or non-natural α-amino acid or β-amino acid residue.

The term “α-amino acid residue” refers to the group “—CHR—NH—” in a moiety represented by the formula: —C(O)—CHR—NH—, and includes natural and non-natural amino acids in either D-configuration or L-configuration. The “α-amino acid” includes α-amino acids in D-configuration, L-configuration, or racemic (D, L) configuration. In the present invention, the amino acid residues have the stereochemistry of normal amino acids, and each amino acid residue can independently exist in the form of either “L” or “D” stereoisomer. Examples of natural or non-natural amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

As used herein, the term “halogen” refers to chlorine, bromine, iodine, or fluorine.

In the definitions above, “optionally substituted” means that a substituent can be further substituted on one or more substituents at any position that is substitutable (including carbon atoms and heteroatoms).

The compounds of the present invention may be in the form of a salt, preferably in the form of a pharmaceutically acceptable salt, or in the form of a solvate.

The term “pharmaceutically acceptable salt” refers to a salt that can be administered to a subject, thereby allowing to provide the compound described herein (directly or indirectly). The salts can be prepared by methods known in the art. Preferably, “pharmaceutically acceptable salts” provide the molecular moieties that are physiologically tolerated and typically do not elicit allergic reactions or similar adverse reactions (for example, nausea, dizziness, etc.), when those salts are administered to humans.

For example, the pharmaceutically acceptable salts of the compounds provided herein are synthesized from the parent compounds comprising a basic or acidic moiety using conventional chemical methods. For example, such salts are generally prepared by reacting the free acid or free base form of these compounds with a base or acid in an appropriate stoichiometric amount in water or an organic solvent, or in a mixture of them. Generally, non-aqueous media such as the ether, ethyl acetate, ethanol, isopropanol, or acetonitrile are preferred. Examples of acid addition salts include mineral acid addition salts (for example, hydrochloride, hydrobromide, hydroiodide, sulfate, nitrate, phosphate), organic acid addition salts, such as acetate, maleate, fumarate, citrate, oxalate, succinate, tartrate, malate, mandelate, methane sulfonate, and p-toluenesulfonate. Examples of alkaline additive salts include inorganic salts (for instance, sodium, potassium, calcium, ammonium, magnesium, aluminum, and lithium salts) and organic alkaline salts (for example, ethylenediamine, ethanolamine, N, N-di-alkylenethanolamine, triethanolamine, glucamine, and salts of basic amino acids).

According to the present invention, the term “solvate” should be understood to mean any form of the active compound of the present invention that is connected through a non-covalent bond to another molecule, which is believed to be a polar solvent in most cases. Examples of such solvates include hydrates and alkoxides, for instance, methoxides.

The compounds of the present invention may be in the crystalline form as either a free compound or a solvate (for example, a hydrate), and it is intended that both forms are within the scope of the present invention. The method of solvation is generally known in the art. An appropriate solvate is a pharmaceutically acceptable solvate. In a specific embodiment, the solvate is a hydrate.

The term “prodrug” as used herein refers to a chemical compound that has undergone chemical derivatization, such as further substitution or addition of chemical moieties, to results in a change in any of its physicochemical properties, for example, solubility or bioavailability, for pharmaceutical applications. For example, the ester derivatives and ether derivatives of the active compound that affords itself after administration to the subject are exemplified. Examples of a well-known method for preparing prodrugs of active compounds in question are familiar for those skilled in the art, and can be found, for example, Krogsgaard-Larsen et al., Textbook of Drug Design and Discovery, Taylor & Francis (April 2002).

The salts, the solvates, and the prodrugs can be prepared by methods known in the art. It will be understood salts, that solvates, or prodrugs that are not pharmaceutically acceptable should still fall within the scope of the present invention since they may be useful in the preparation of the salts, solvates or prodrugs that are pharmaceutically acceptable.

As mentioned above, the phosphoramidate-activated AMP (ATP prodrug) developed in this study is a prodrug that exerts its effects by being metabolized within the cells, converting from AMP to ATP, thereby increasing the intracellular ATP concentration. Various prodrugs that exert pharmacological effects through similar intracellular metabolism have been reported to date. For example, the antiviral medicament Remdesivir which is used for Ebola virus infection and coronavirus infection (GS-5734 exerts its therapeutic effect by being triphosphorylated within the cells (inhibition of viral replication) (ACS CentSci. 2020, 6, 5, 672-683).

The expected effects of the medicaments (prodrugs) that can increase the intracellular ATP levels are as follows:

### 1. To Bring Anti-Aging Effects to the Cells, Tissues, Organs, and Individuals by Compensating for the Decrease in ATP Levels Due to Aging.

It has been reported that the intracellular ATP levels decrease with aging (PNAS, 2006, 103, 1727). ATP supplementation can reduce oxidative stress damage to the cells.

### 2. Treatment of Neurodegenerative Diseases Such as Parkinson's Disease, Alzheimer's Disease, and ALS

Parkinson's disease is thought to be related to the decrease in ATP due to impaired mitochondrial function (EBioMedicine, 2017, 22, 225). ATP supplementation can provide an effective medicament, and can be effect against the onset of ALS due to abnormal intracellular liquid-liquid phase separation.

### 3. Effects on the Maintenance of Cell Survival Rates and Functions in Three-Dimensional Culture by Supplementing ATP Deficiency Due to Hypoxia

When oxygen is deficient, the amount of ATP production significantly decreases, leading to cell necrosis, and therefore, ATP supplementation can suppress onset.

### 4. Effects of ATP Supplementation in Tissue Regeneration

There are reports of delivering ATP encapsulated in liposomes (The American Journal of Surgery (2010) 199, 823-832).

ATP supplementation to areas of skin and other tissue in ATP deficiency can promote tissue regeneration.

As mentioned above, ATP supplementation may be artificially conducted, and if its effects are confirmed, various applications can be expected. The present invention proposes a novel ATP therapy.

5. Therapeutic Effects on Non-Alcoholic Fatty Liver Diseases and/or Hepatitis

It has been often reported that, in non-alcoholic fatty liver diseases and/or hepatitis (NAFLD and/or NASH), the mitochondria of liver cells are impaired, leading to a decrease in intracellular ATP levels and a worsening of the pathological conditions. Improvement of the decreased ATP level in such liver diseases would provide any expectation that the potential for application in the prevention and treatment of NAFLD and/or NASH diseases.

### 6. Effect on AMPK Activation

It has been reported that low molecular weight drugs known as anti-aging drugs have the effect of activating AMPK. For example, metformin (B. Onken et al., PLOS ONE, 2010) and resveratrol (JG Wood et al., Nature, 2004) are exemplified. The ATP prodrugs of the present invention have been shown to function as an AMP-activated protein kinase (AMPK) activator, indicating that the compounds of the present invention activate AMPK, increase intracellular ATP levels, and consequently exhibit anti-aging effects.

### Pharmaceutical Compositions

In another aspect, the present invention relates to a pharmaceutical composition comprising the compound of the present invention, or a pharmaceutically acceptable salt or solvate thereof.

In one embodiment of this aspect, the present invention relates to a pharmaceutical composition comprising the compound of the present invention, or a pharmaceutically acceptable salt or solvate thereof. Specifically, it is a pharmaceutical composition for treating or preventing a state of decreased intracellular ATP levels.

As used herein, the term “treat” refers to reversing, alleviating, inhibiting the progression of, or preventing a disorder or condition to which such a term applies, or one or more symptoms of such a disorder or condition, unless otherwise specified. In one embodiment, the term “treatment” refers to the administration of the compound or composition of the present invention to alleviate or eliminate the symptoms of a state of decreased intracellular ATP levels and/or to reduce the state of decreased intracellular ATP levels in an individual. The treatment may be conducted as a preventive measure before the onset of the disease or condition, or it may also be conducted after the onset of the disease.

“Preventing” refers to any action taken to ensure that the clinical symptoms of a disease or condition do not occur. The term “prevention” also encompasses the administration of the compounds or compositions of the present invention in therapeutic effective amounts to prevent the onset of disease symptoms and/or to prevent reaching a state of decreased intracellular ATP levels (for example, pre-exposure prevention).

In the present invention, a pharmaceutical composition generally refers to a drug for the treatment or prevention of diseases, or for testing and diagnosis.

The pharmaceutical composition of the present invention can be formulated by methods known to those skilled in the art. For example, the composition can be used orally or non-orally in the form of a sterile solution or suspension with water or any other pharmaceutically acceptable liquid, as well as in the form of an injectable preparation for non-oral use. For example, it is understood that the composition may be formulated by mixing with an acceptable carrier or medium in pharmacology, specifically, sterilized water, saline solution, vegetable oil, emulsifiers, suspending agents, surfactants, stabilizers, flavoring agents, excipients, vehicles, preservatives, binders, and so on, as appropriate, leading to a unit dosage form required for generally accepted pharmaceutical practices. The amount of active ingredient in these formulations is set to obtain an appropriate dosage within the specified range.

Sterile compositions for injection can be formulated according to standard preparation methods using a vehicle such as injection-grade distilled water.

Examples of injectable aqueous solutions include isotonic solutions such as physiological saline, glucose, and other adjunctive medications (such as D-sorbitol, D-mannose, D-mannitol, and sodium chloride). It is acceptable to use appropriate dissolution auxiliaries, such as alcohols (e.g., ethanol), polyalcohols (such as propylene glycol, polyethylene glycol), and non-ionic surfactants (such as Polysorbate 80™, HCO-50) in combination.

Examples of oily liquids include sesame oil and soybean oil, and it is acceptable to use benzyl benzoate and/or benzyl alcohol as solubilizing agents in combination. Additionally, buffer agents (such as phosphate buffer solution and sodium acetate buffer solution), anesthetic agents (such as procaine hydrochloride), stabilizers (such as benzyl alcohol and phenol), and antioxidants may be combined. The prepared injection solution is usually filled into a suitable ampoule.

The pharmaceutical composition of the present invention is orally or non-orally administered. For example, the composition can be in injection type, intranasal administration type, pulmonary administration type, and transdermal administration type. For example, the composition can be administered systemically or locally through intravenous injection, intramuscular injection, intraperitoneal injection, or subcutaneous injection.

The method of administration can be appropriately selected based on the patient's age and symptoms. The dosage of the pharmaceutical composition can be set, for example, in the range of 0.0001 mg to 1000 mg per 1 kg of body weight per administration. Alternatively, for example, the dosage can be from 0.001 to 100,000 mg per patient, but the present invention is not necessarily limited to these values. The dosage and administration method may vary depending on the patient's weight, age, symptoms, and other factors, and those skilled in the art can design an appropriate dosage and administration method, taking account of those conditions.

In another aspect, the present invention relates to a method for treating or preventing states of decreased intracellular ATP levels, such as aging, non-alcoholic fatty liver disease and/or hepatitis, mitochondrial diseases, cancers and hypoxic conditions due to insufficient blood flow, inflammatory tissue regeneration areas, or neurodegenerative diseases, which comprises administering the compounds of the present invention to subjects in need of such treatment, preferably such a method comprising administrating an effective amount of the compounds of the present invention to such subjects.

Further, in a different aspect, the present invention relates to the compounds of the present invention for treating or preventing states of decreased intracellular ATP levels, such as aging, non-alcoholic fatty liver disease and/or hepatitis, mitochondrial diseases, cancers and hypoxic conditions due to insufficient blood flow, inflammatory tissue regeneration areas, or neurodegenerative diseases.

In a further different aspect, the present invention relates to the use of the compounds of the present invention for the manufacture of a medicament for treating or preventing states of decreased intracellular ATP levels, such as aging, non-alcoholic fatty liver disease and/or hepatitis, mitochondrial diseases, cancers and hypoxic conditions due to insufficient blood flow, inflammatory tissue regeneration areas, or neurodegenerative diseases.

Hereinafter, the present invention will be described in more detail by way of the working examples, but it should be noted that these would not limit the scope of the present invention and merely illustrate the invention.

### EXAMPLES

### Example 1

### Phosphoramidate AMP (ATP Prodrug)

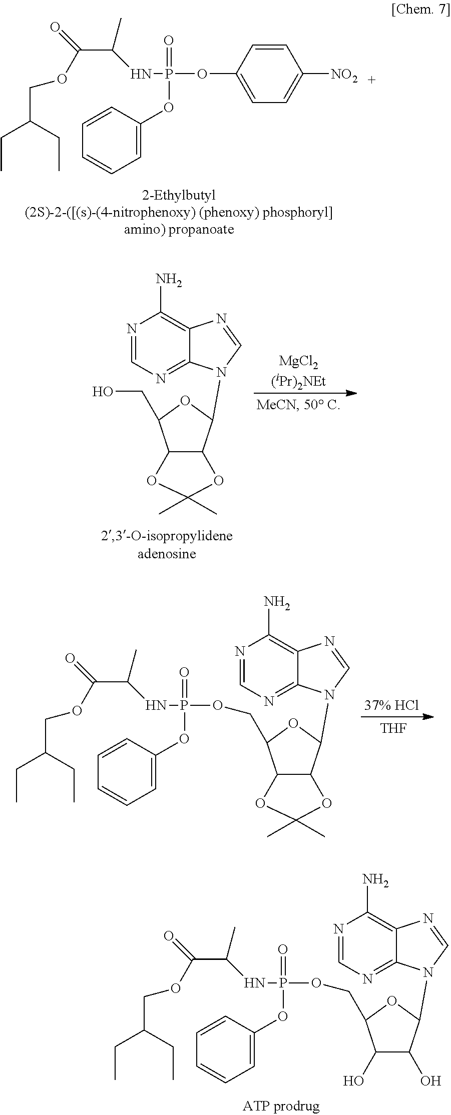 [SMILES: CCC(CC)COC(=O)C(C)NP(=O)(Oc1ccccc1)Oc1ccc([N+](=O)[O-])cc1 ; CC1(C)OC2C(CO)OC(n3cnc4c(N)ncnc43)C2O1 ; CCC(CC)COC(=O)C(C)NP(=O)(OCC1OC(n2cnc3c(N)ncnc32)C2OC(C)(C)OC12)Oc1ccccc1 ; "THF" ; CCC(CC)COC(=O)C(C)NP(=O)(OCC1OC(n2cnc3c(N)ncnc32)C(O)C1O)Oc1ccccc1]

### Example 1-1: (S)-2-Ethylbutyl 2-(((S)-(((2R,3S,4R,5R)-5-(6-amino-9H-purine-9-yl)-2,2-dimethyltetrahydro-3,4(4-(1,3-dioxol-2-yl)methoxy)(phenoxy)phosphoryl)amino)propanoate

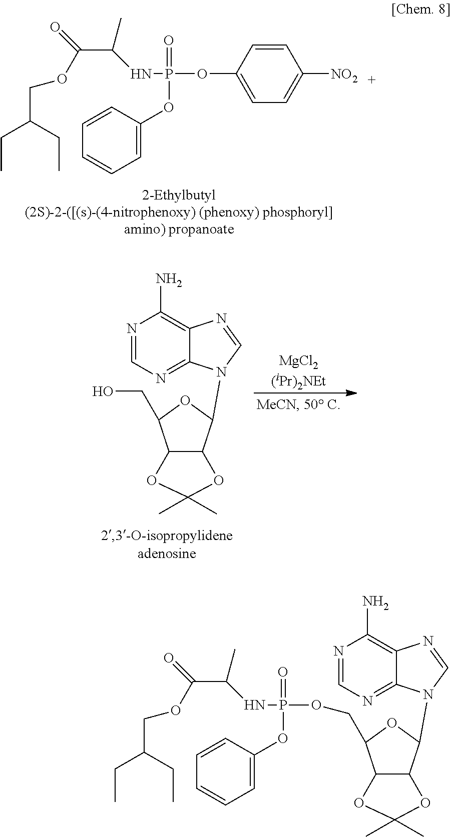 [SMILES: CCC(CC)COC(=O)C(C)NP(=O)(Oc1ccccc1)Oc1ccc([N+](=O)[O-])cc1 ; CC1(C)OC2C(CO)OC(n3cnc4c(N)ncnc43)C2O1 ; CCC(CC)COC(=O)C(C)NP(=O)(OCC1OC(n2cnc3c(N)ncnc32)C2OC(C)(C)OC12)Oc1ccccc1]

2-Ethylbutyl (2s)-2-([(s)-(4-nitrophenoxy)(phenoxy) phosphoryl]amino)propanoate (1.8 g, 4 mmol) obtained from Combi-Blocks and 2′,3′-O-isopropylideneadenosine (1.0 g, 3.3 mmol) obtained from Tokyo Chemical Industry were reacted 50° C. in the presence of magnesium chloride and N, N-diisopropylethylamine in 20 mL of acetonitrile. After purification with the silica gel column, the compound in the title (1.9 g, 92%) was obtained.

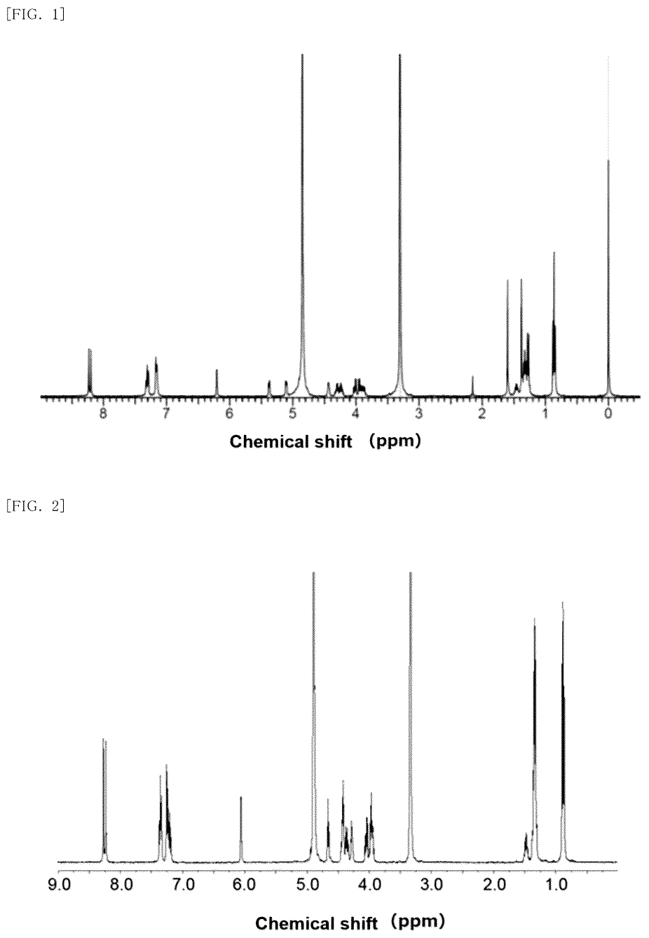

The chart of the NMR spectrum of the obtained compound is shown in FIG. 1.

### Example 1-2: Phosphoramidate AMP (ATP Prodrug)

### (S)-2-Ethylbutyl 2-(((S)-(((2R,3S,4R,5R)-5-(6-amino-9H-purine-9-yl)-3,4-dihydroxy-tetrahydrofuran-2-yl)methoxy) (phenoxy)phosphoryl)amino)propanoate

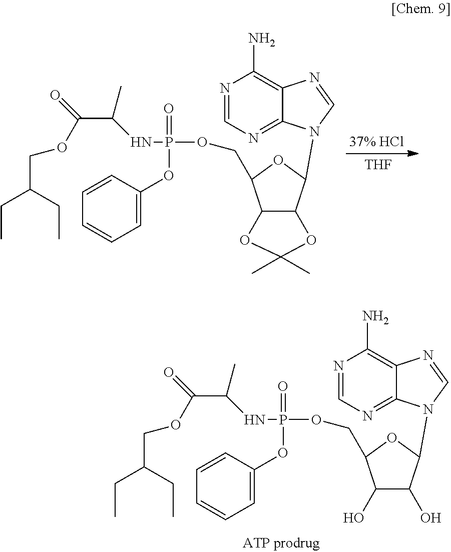 [SMILES: CCC(CC)COC(=O)C(C)NP(=O)(OCC1OC(n2cnc3c(N)ncnc32)C2OC(C)(C)OC12)Oc1ccccc1 ; "THF" ; CCC(CC)COC(=O)C(C)NP(=O)(OCC1OC(n2cnc3c(N)ncnc32)C(O)C1O)Oc1ccccc1]

The compound prepared in Example 1-1 (1.2 g, 2 mmol) was reacted with 2 mL of 37% hydrochloric acid at 0° C. in THE (10 mL), and after purification by silica gel column chromatography, the compound in the title (1.5 g, 75%, melting point 129.9° C.) was obtained.

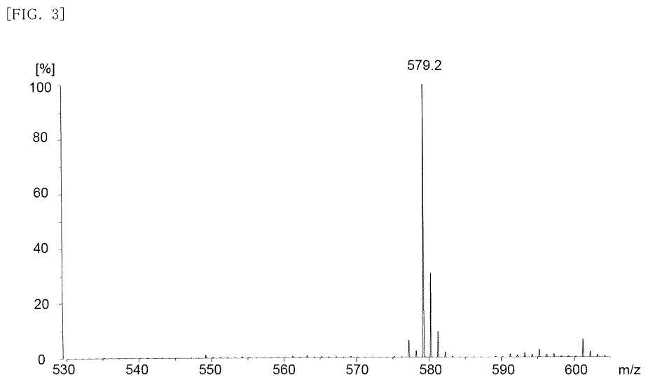

The charts of the NMR spectrum and the FAB MS spectrum of the obtained compounds are shown in FIGS. 2 and 3, respectively.

### Example 2

### Phosphoramidate AMP (ATP Prodrug-Neo)

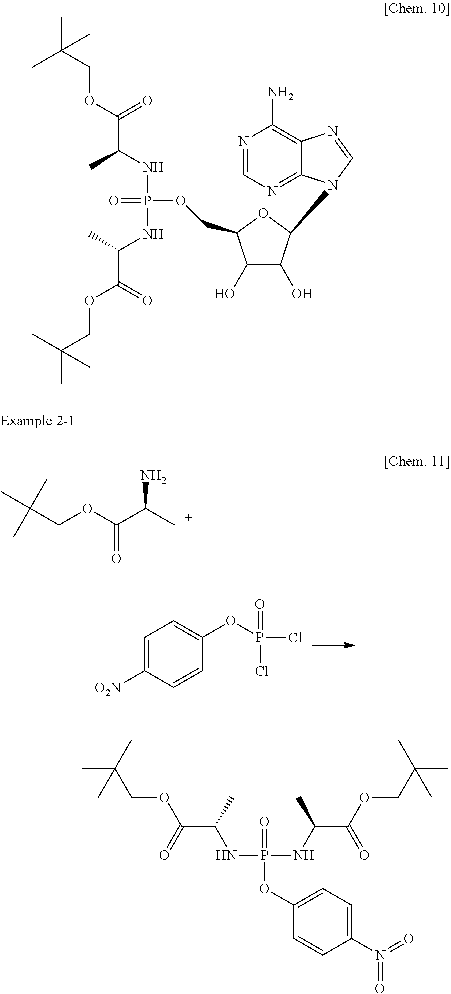

4-Nitrophenyl phosphorodichloridate purchased from MERCK The reaction was carried out at room temperature in dichloromethane with the presence of triethylamine, using 5.1 mmol of L-alanine neopentyl ester hydrochloride purchased from ACROTEIN (2.0 g, 10.2 mmol). After purification with the silica gel column, the desired compound was obtained (0.8 g, 30%).

### Example 2-2

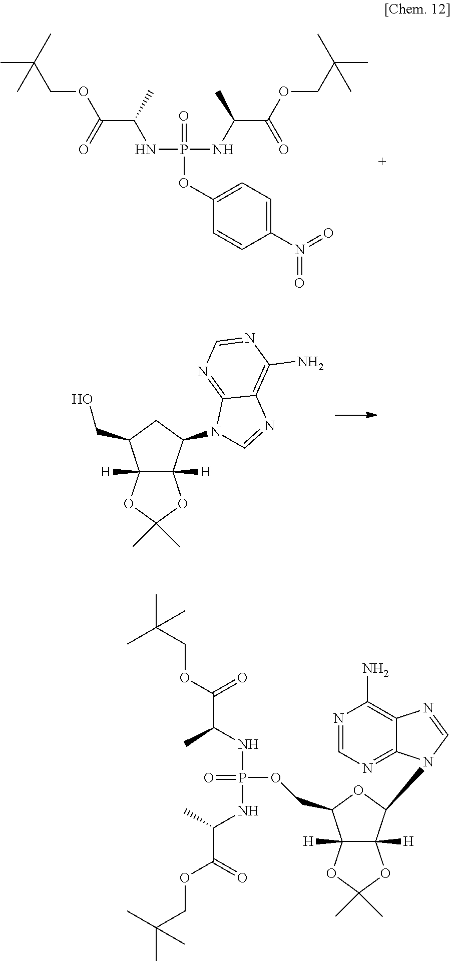

The compound prepared in Example 2-1 (0.6 g. 1.2 mmol was reacted with 2′,3′-O-isopropylidene adenosine (1.0 g, 3.3 mmol) purchased from Tokyo Chemical Industry in the presence of magnesium chloride, N,N-diisopropylethylamine in acetonitrile (20 mL) at 50° C. After purification with the silica gel column, the desired compound (0.5 g, 79%) was obtained.

### Example 2-3

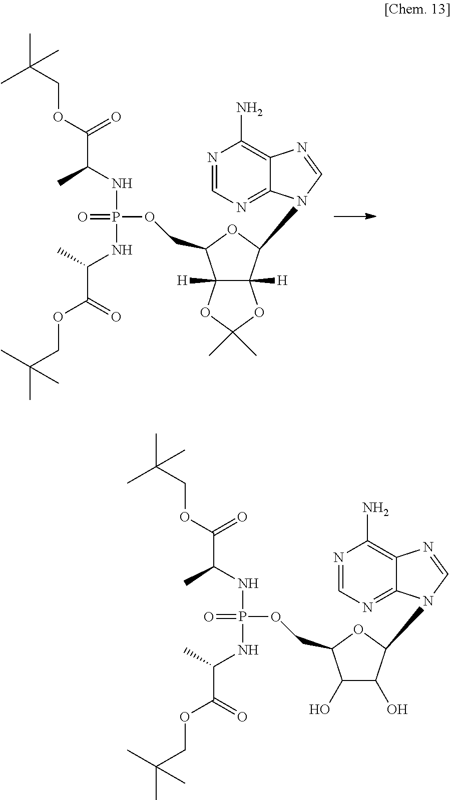 [SMILES: C[C@H](NP(=O)(N[C@@H](C)C(=O)OCC(C)(C)C)OC[C@H]1O[C@@H](n2cnc3c(N)ncnc32)[C@@H]2OC(C)(C)O[C@@H]21)C(=O)OCC(C)(C)C ; C[C@H](NP(=O)(N[C@@H](C)C(=O)OCC(C)(C)C)OC[C@H]1O[C@@H](n2cnc3c(N)ncnc32)C(O)C1O)C(=O)OCC(C)(C)C]

The compound prepared in Example 2-2 (0.53 g, 0.79 mmol) was reacted was reacted with 2 mL of 37% hydrochloric acid at 0° C. in THE (5 mL), and after purification with silica gel column, the desired compound (0.1 g, 22%) was obtained.

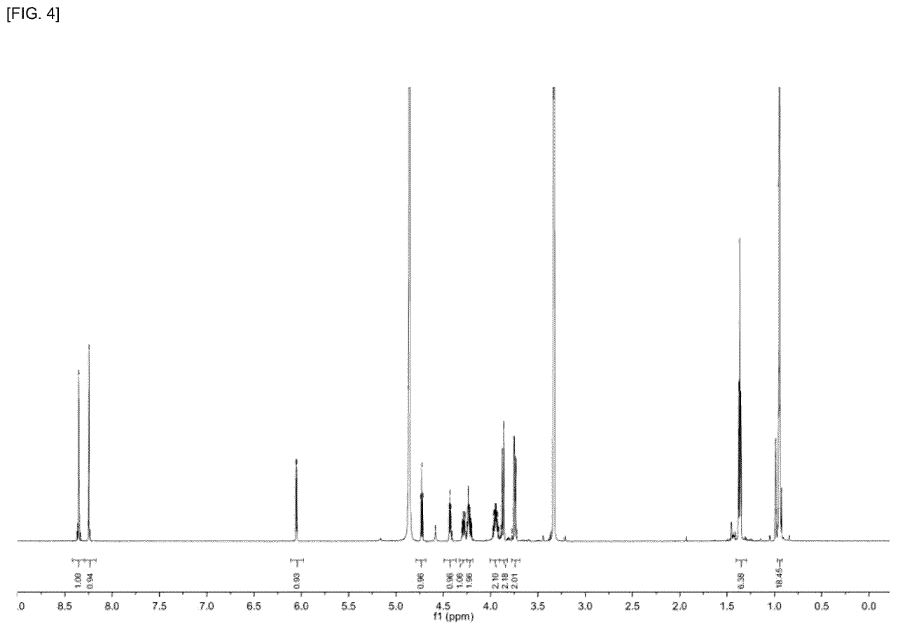

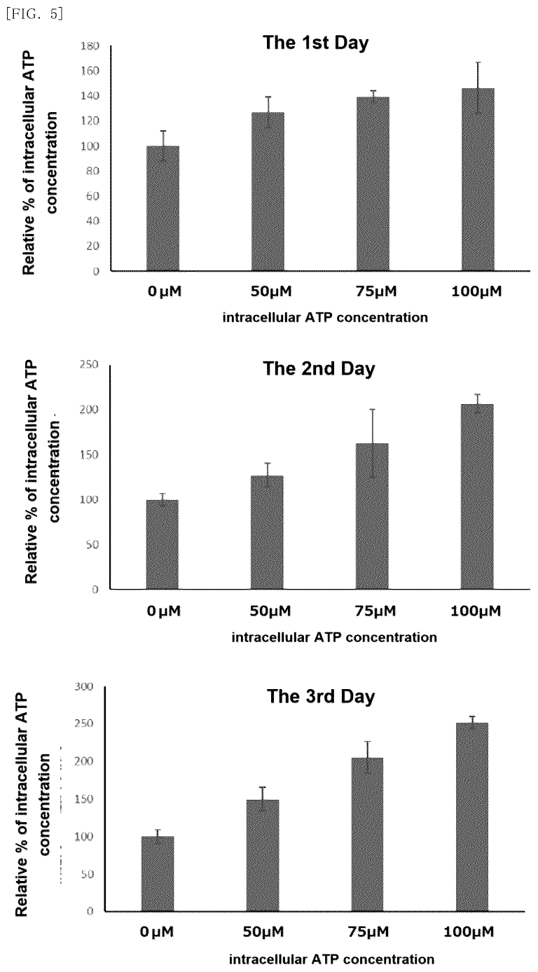

The chart of the NMR spectrum of the obtained compound is shown in FIG. 4.

### Test Example 1

### Effect of ATP Prodrugs on Intracellular ATP Concentration

MCF7 (human breast cancer cells) (RIKEN BRC) were seeded at 5000 cells per well in DMEM low glucose, 10% FBS, and 1% penicillin/streptomycin, and, after 3 hours, solutions of the ATP prodrug prepared in Example 1 in DMSO at varying concentrations were added to those wells. Three days later, the amounts of DNA were measured using picogreen, and converted to cell counts from a calibration curve separately created. The amount of ATP was quantified using the ATP measurement kit (Dojin Chemical), and the amount of ATP per cell was calculated.

The results obtained are shown in FIG. 5. FIG. 5 shows that the intracellular ATP amount increased in an ATP prodrug concentration-dependent manner, and the intracellular ATP amount reached approximately 2.5 times compared to the control level after 3 days (when 100 μM of the ATP prodrug was added). In this way, the prodrug form of ATP facilitated to increase the intracellular ATP concentration.

### Test Example 2

### Effect of ATP Analogs on Intracellular ATP Concentration

Adenosine, AMP, ATP, and the ATP prodrug were added to the culture medium of MCF7 cells (5000 cells/well) at a concentration of 100 μM, and, one day and two days later, ATP was measured, followed by normalizing the results to the DNA amounts.

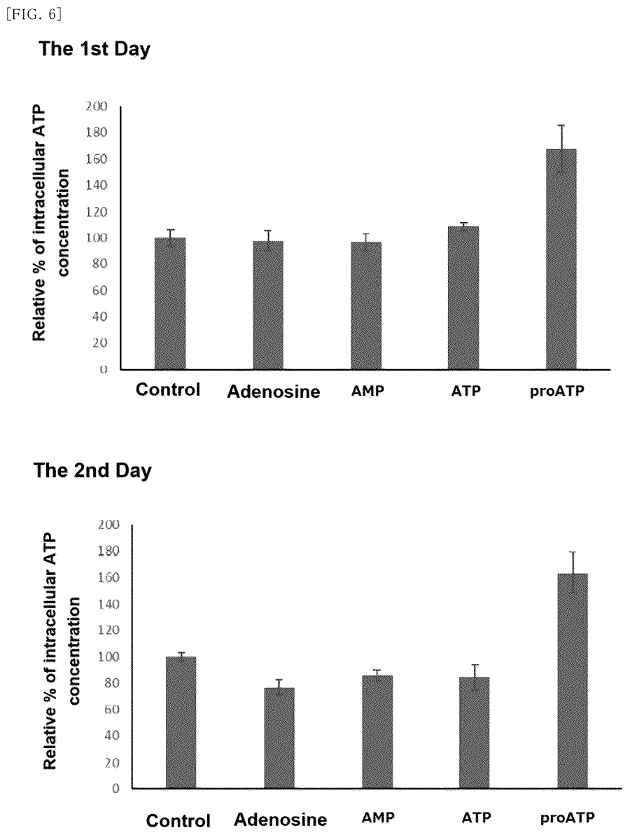

The results obtained are shown in FIG. 6. FIG. 6 shows that the addition of the ATP prodrug only increased the intracellular ATP level.

### Test Example 3

### Effect on the Intracellular ATP Concentration Using Normal Cells

Human fibroblasts (NHDF) (Takara Bio) were seeded at 5000 cells per well in DMEM low glucose, 10% FBS, and 1% penicillin/streptomycin, and, after 3 hours, a solution of the ATP prodrug prepared in Example 1 in DMSO at varying concentrations was added to the wells. One day later, the amount of DNA was measured using picogreen, and converted to cell count from a calibration curve separately created. The ATP level was quantified using the ATP measurement kit (Dojin Chemical), and the ATP level per cell was calculated.

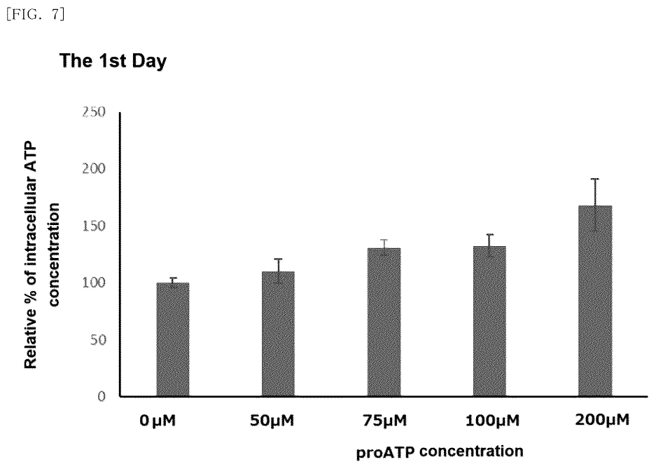

The results obtained are shown in FIG. 7. FIG. 7 shows that even in normal cells NHDF, the addition of the ATP prodrug increases the intracellular ATP levels in a concentration-dependent manner, and the level reached up to 1.7 times compared to the control (0 μM) at a concentration of 200 μM of the ATP prodrug.

### Test Example 4

### Effects on Increased ATP and Stress Load on in Aging Cells

Human Dermal Fibroblasts (HDF) derived from 75-year-old and 29-year-old human (Toyobo) were seeded at 5000 cells/well in DMEM low glucose, 10% FBS, and 1% penicillin/streptomycin, and three hours later, the ATP prodrug dissolved in DMSO was added at either 0 or 50 μM. (A) The sample was incubated for 24 hours. The amount of DNA was measured using picogreen, and converted to cell count from a calibration curve separately created. The ATP level was quantified using the ATP measurement kit (Dojin Chemical), and the ATP level per cell was calculated. (B) 75 μM of the ATP prodrug was added to the HDF derived from 75-year-old human, and the medium was replaced with serum-free medium comprising 200 μM of hydrogen peroxide after 24 hours, followed by incubating the mixture for 6 hours. After 6 hours, the cells were washed, and the cell numbers were compared using the WST-8 kit.

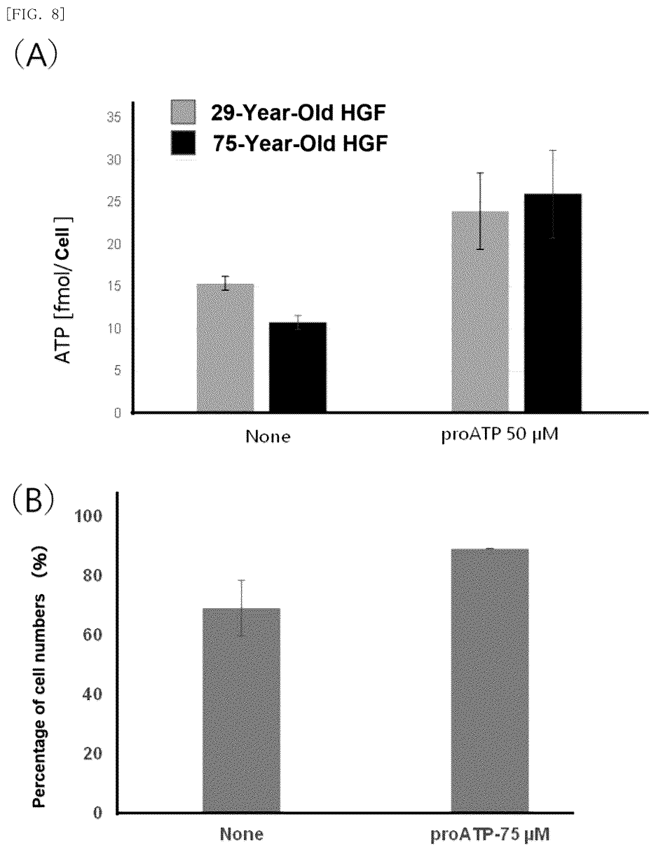

The results obtained are shown in FIG. 8. FIG. 8 (A) shows that the intracellular ATP levels increased with the addition of the ATP prodrug in cells derived from 29-year-old and 75-year-old human. FIG. 8 (B) shows that, in cells derived from a 75-year-old human, the number of cells decreases under stress conditions in the absence of the ATP prodrug, while the addition of the ATP prodrug at 75 μM suppresses the decrease in cell number. In this way, it was suggested that even in aged cells, the addition of ATP prodrugs increased the intracellular ATP concentration, leading to improved stress tolerance.

### Test Example 5

### Cytotoxicity of ATP Prodrugs

NHDF (normal human dermal fibroblasts) were seeded at 5000 cells per well in DMEM/F12 with 10% FBS and 1% penicillin/streptomycin, and after 3 hours, a solution of the ATP prodrug prepared in Example 1 in DMSO at varying concentrations was added to the wells. After 24 hours, the release of the cytotoxicity marker, lactate dehydrogenase (LDH), was quantified using the LDH assay kit (Dojin Chemical), and the cytotoxicity was calculated.

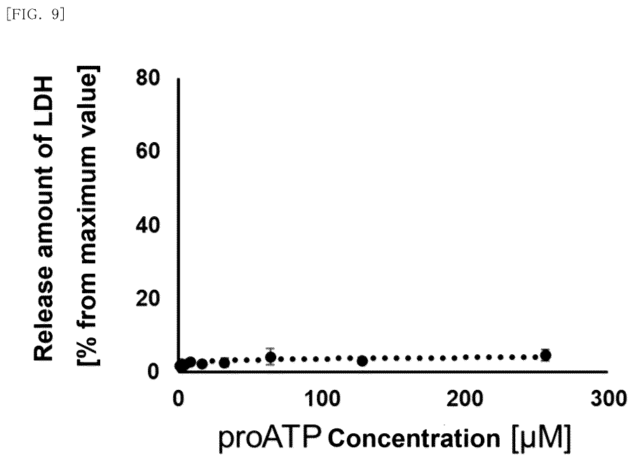

The results obtained are shown in FIG. 9. FIG. 9 shows that even when the ATP prodrug is added at a high concentration (256 μM), the amount of LDH release remains low. In this way, the ATP prodrug was confirmed to have low cytotoxicity to cells.

### Test Example 6

### Effect of ATP Prodrugs on Liver Cells

Human liver cancer cells (HepG2) were seeded at 5000 cells per well in DMEM with 10% FBS and 1% penicillin/streptomycin, and after 3 hours, a solution of the ATP prodrug prepared in Example 1 in DMSO at varying concentrations was added to the wells. After 24 hours, the amount of DNA was measured using picogreen, and converted to cell count based on a calibration curve separately created. The ATP level was quantified using the ATP measurement kit (Dojin Chemical), resulting in the ATP level per cell.

In addition to this study, normal pig liver cells were seeded at 5000 cells per well in the liver cell differentiation environment (Hepatp-STIM, 1 μg/100 mL EGF, 1% penicillin/streptomycin), and after 3 hours, a solution of the ATP prodrug in DMSO at varying concentrations was added to the wells. The DNA amount was measured using picogreen 24 hours later. The ATP level was quantified using the ATP measurement kit (Dojin Chemical), and the ATP level was provided.

The results obtained are shown in FIG. 10. It was confirmed that the ATP prodrug increased in intracellular ATP levels in two types of liver cells (cultured cells and primary cells).

### Test Example 7

### Evaluation of Intracellular ATP Levels Using AMPK Inhibitors

100 μL of serum medium (DMEM/F12, 10% FBS) was added to the 96-well plate, and preconditioning was performed in the incubator for 1 hour. Subsequently, normal human dermal fibroblasts were seeded (cell seeding number: 5×10³ cells/well). Three hours after the seeding, the ATP prodrug prepared in Example 1 (final concentration: 100 μM) and the AMP-activated protein kinase (AMPK) inhibitor Dorsomorphin (final concentrations of 0 or 12.5 μM) were added to each well, and the mixtures were cultured at 37° C. for 24 hours. 100 μL of the detection reagent included in the ATP measurement kit was added to each well, and the color reaction was performed in the dark at room temperature for 30 minutes. Then, 50 μL of the reaction quenching solution was added to each well, and the absorbance at 490 nm was measured using the microplate reader.

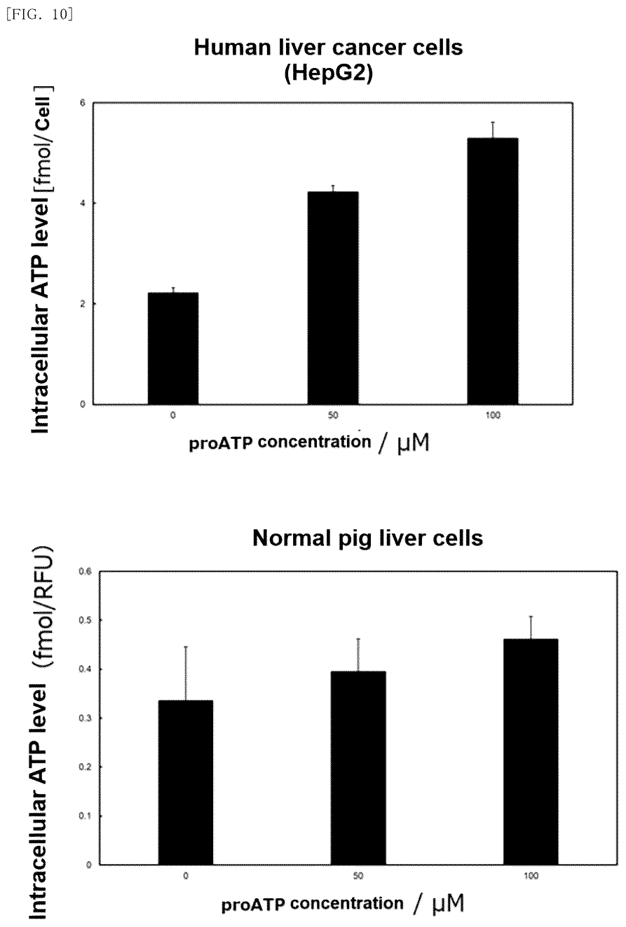

The results obtained are shown in FIG. 11. The addition of the AMPK inhibitor Dorsomorphin inhibited a portion of the ATP production due to the ATP prodrug. This result suggested that the ATP prodrug activates the AMPK, increases the intracellular ATP levels, and demonstrates anti-aging effects.

### Test Example 8

### Confirmation of Lifespan Extension Effect Using Model Organism (Nematode): Part 1

### Preparation of Substrate

The ATP prodrug prepared in Example 1, dissolved in DMSO, was added to the liquid Nematode Growth Medium (NGM) that had been sterilized and maintained at 60° C. at a final concentration of 100 μM, so as to prepare an ATP prodrug-containing NGM plate. At this time, the final concentration of DMSO in the NGM was adjusted to 1%. The NGM plate that did not comprise any ATP prodrug was also added with DMSO to achieve a final concentration of 1% DMSO. Onto the surface of the NGM plate used immediately before survival analysis, the bacterium Escherichia coli strain OP50 stirred in M9 buffer was applied as food for the nematodes.

### Survival Analysis

Thirty-five nematodes were transferred to the NGM plate, after performing age synchronization. The nematodes were transferred to a new NGM plate every day from the first day of adulthood (the third day) to the seventh day, and every two days from the eighth day until the nematodes died. At the same time, the numbers of living nematodes and dead nematodes were recorded. Survival status of the nematodes was determined by whether they reacted when touched. Average lifespan was calculated from the results obtained, and the average lifespans were compared to determine the lifespan extension rate of the nematodes. This experiment was conducted four times to confirm the reproducibility (In cases of both with and without the addition of proATP, n=114).

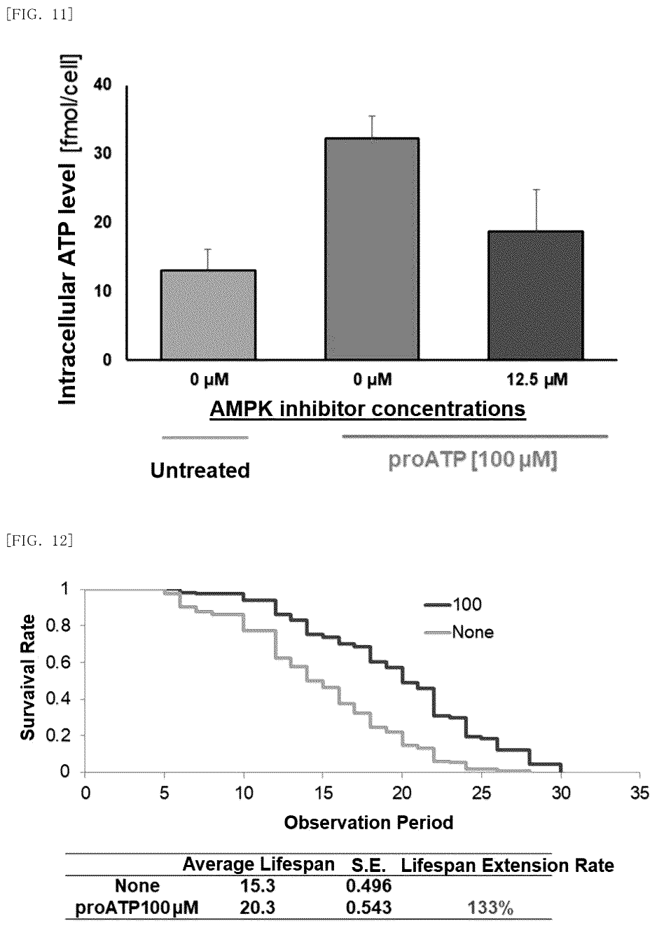

The results obtained are shown in FIG. 12. FIG. 12 shows that the nematodes cultured on the ATP prodrug-containing NGM plate had an average lifespan extended by approximately 33% compared to the control. In this way, the addition of the ATP prodrug to the nematodes facilitated to extend the average lifespan of the nematodes.

For reference, a table comparing the effects of other drugs known to extend the lifespan of the nematode is presented.

TABLE 1

| | Lifespan | |
|---|---|---|
| Drugs | Extension Rate | References |
| Nicotinamide | 116% | L. Mouchiroud et al., Cell, 2013 |
| Metformin | 127% | B. Onken et al., PLOS ONE, 2010 |
| Rapamycin | 126% | D. Chen et al., Cell Rep, 2013 |
| Resveratrol | 110% | J G Wood et al., Nature, 2004 |
| proATP | 133% | the present invention |

### Comparison of Intracellular ATP Levels in Model Organism (Nematode)

The culture of the nematodes and the addition of the ATP prodrug to the nematodes were conducted under the same conditions as those in the survival analysis. On the seventh day of cultivation, five nematodes were collected for each condition, totaling twenty-five nematodes, and were transferred to a 24-well plate containing PBS, followed by conducting ultrasonic disruption while kept on ice for one minute. Then, the crushed liquid was collected into a microtube, and after centrifugation (15,000 rpm, 30 minutes, 4° C.), the supernatant was collected. Amount of the ATP in the supernatant was quantified using the ATP measurement kit (Dojin Chemical), and the amount of the ATP per nematode was calculated.

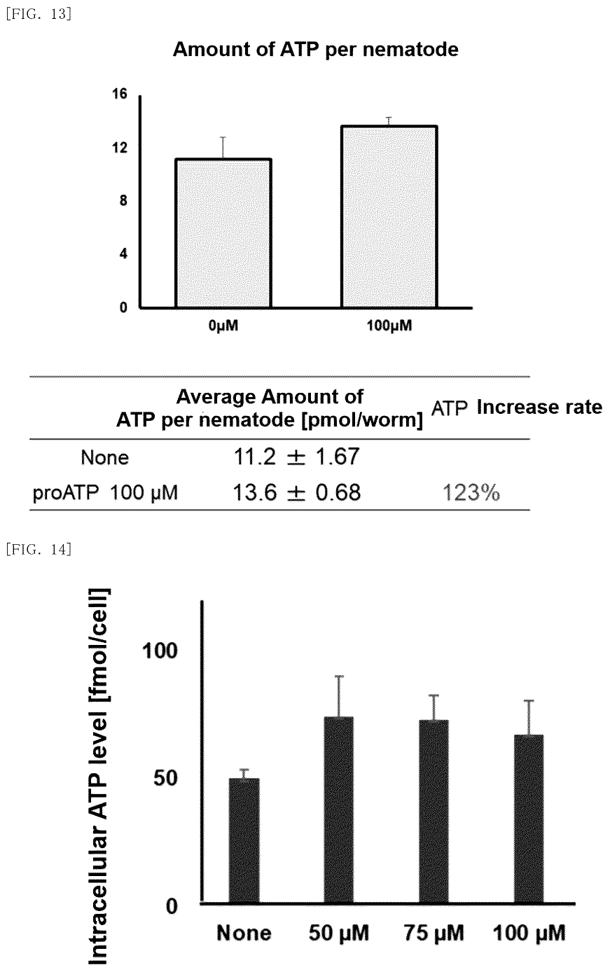

The results obtained are shown in FIG. 13. FIG. 13 shows that the amount of ATP per nematode cultured on the ATP prodrug-containing NGM plate was approximately 23% higher than that of the control. In this way, the addition of the ATP prodrug to the nematodes facilitated to increase the amount of ATP within the nematodes.

### Test Example 9

### Effect of ATP Prodrug-Neo on Intracellular ATP Concentration

HDF75 (human dermal fibroblasts derived from a 75-year-old) (Cell Applications, Inc.) were seeded at 5000 cells per well in the human dermal fibroblast growth medium, and after 3 hours, a solution of the ATP prodrug-neo prepared in Example 2 in DMSO at varying concentrations was added to the well. After 24 hours, the amount of DNA was measured using the picogreen and converted to cell count from a calibration curve separately created. The amount of ATP was quantified using the ATP measurement kit (Dojin Chemical), and the amount of ATP per cell was calculated.

The results obtained are shown in FIG. 14. FIG. 14 shows that the intracellular ATP levels increased due to the ATP prodrug-neo. In this way, the ATP prodrug with the different structure from the ATP prodrug of Example 1 also was able to increase the intracellular ATP concentration.

### Test Example 10

### Confirmation of Lifespan Extension Effect Using Model Organism (Nematode): Part 2

### Preparation of Substrate

ATP prodrug-neo dissolved in DMSO, was added to the liquid Nematode Growth Medium (NGM) that had been sterilized and maintained at 60° C. at a final concentration of 200 UM, so as to prepare an ATP prodrug-neo-containing NGM plate. At this time, the final concentration of DMSO in the NGM was adjusted to 1%. The NGM plate that did not comprise any ATP prodrug-neo was also added with DMSO to achieve a final concentration of 1% DMSO. Onto the surface of the NGM plate used immediately before survival analysis, the Escherichia coli strain OP50 stirred in M9 buffer was applied as food for the nematodes.

### Survival Analysis

Thirty-five nematodes were transferred to the NGM plate, after performing age synchronization. The nematodes were transferred to a new NGM plate every day from the first day of adulthood (the third day) to the seventh day, and every two days from the eighth day until the nematodes died. At the same time, the numbers of living nematodes and dead nematodes were recorded. Survival status of the nematode was determined by whether they reacted when touched. Average lifespan was calculated from the results obtained, and the average lifespans were compared to determine the lifespan extension rate of the nematodes. This experiment was conducted four times to confirm the reproducibility.

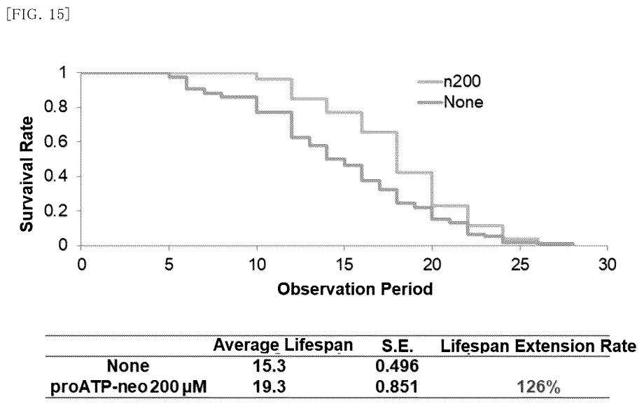

The results obtained are shown in FIG. 15. FIG. 15 shows that the nematodes cultured on the ATP prodrug-neo containing NGM plate had an average lifespan extended by approximately 26% compared to the control. In this way, the addition of the proATP-neo to the nematodes facilitated to extend the average lifespan of the nematodes.

### INDUSTRIAL APPLICABILITY

As described above, the ATP prodrug of the present invention should be a groundbreaking substance that can artificially increase intracellular ATP levels. Until now, there have been no reports of ATP precursors that can increase the concentration of ATP in this manner. Furthermore, ATP is a universal energy currency substance present in all living organisms, and the present invention that can control this concentration provides extremely high impact on applicability in pharmaceuticals.

1. A compound represented by formula (I): 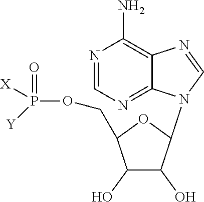
in which
X and Y are the same or different and each represents a substituent selected from Substituent group A represented below, or a substituent selected from Substituent groups B and C represented below, provided that X and Y are not simultaneously any substituent selected from Substituent group A;
Substituent group A: This is the case when the atom connected to the phosphorus atom is oxygen; a group represented by formula 1: 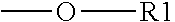
in which R1 is selected from the group consisting of a hydrogen, a C1 to C8 alkyl group, a C1 to C3 alkylaryl group, CH₃OCH₂—, CH₃OCH₂CH₂—, an ester group, a phenyl group, a pyridyl group, a benzyl group, an indolyl group, and a naphthyl group, wherein the aromatic ring or heterocyclic ring thereof may be substituted with one to three of the following functional groups: halogen, C1-C6 alkyl, C2-C6 alkenyl, C1-C6 halogenated alkyl, C1-C6 alkoxy, C1-C6 halogenated alkoxy, phenyl, hydroxy C1-C6 alkyl, C3-C6 cycloalkyl, C1-C6 alkyl carbonyl, C3-C6 cycloalkyl carbonyl, carboxy C1-C6 alkyl, hydroxyl group, amino group, cyano group, nitro group, trimethylsilyl group;
Substituent group B: This is the case when the atom connected to the phosphorus atom is nitrogen; a group represented by formula 2: 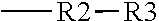
in which R2 is a residue collectively referred to as an aminocarboxylic acid;
R3 is a straight-chain C1 to C6 alkyl group or a branched or cyclic C3 to C6 alkyl group that binds to the carboxylic acid part of the aminocarboxylic acid in R2, preferably, for example, methyl, ethyl, n-propyl, isopropyl, cyclopropyl, n-butyl, isobutyl, sec-butyl, tert-butyl, n-pentyl, isopentyl, sec-pentyl, tert-pentyl, neopentyl, 1-methylpentyl, n-hexyl, isohexyl, sec-hexyl, tert-hexyl, neohexyl, and the like; a C4 to C8 cycloalkyl group that may be substituted with a C1 to C8 alkyl group; a tetrahydropyranyl group; a benzyl group that may be substituted with a halogen, a C1 to C8 alkyl group, or a C1 to C6 alkoxy group; a 2-phenylethyl group that may be substituted with a halogenated phenyl group; and an indole group; and
Substituent group C: This is the case when the atom connected to the phosphorus atom is nitrogen; C3 to C5 heterocyclic rings, including a morpholine ring, a piperazine ring, a thiomorpholine ring, or a cyclic structure represented by formula 3: 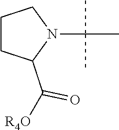
in which R4 is a hydrogen, a C1 to C8 alkyl group that may be substituted with a C3 to C6 cycloalkyl group: a C4 to C8 cycloalkyl group: a tetrahydropyranyl group: a benzyl group that may be substituted with a halogen, a C1 to C8 alkyl group, or a C1 to C6 alkoxy group: a 2-phenylethyl group that may be substituted with a halogenated phenyl group, or an indole group;
or a pharmaceutically acceptable salt or solvate thereof.

2. The compound according to claim 1, in which, in Substituent group A, a phenyl group that may be substituted with halogen, C1-C6 alkyl, or C1-C6 alkoxy;
or a pharmaceutically acceptable salt or solvate thereof.

3. The compound according to claim 1, in which, in Substituent group B, the substituent is a group represented by formula 2: 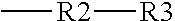
in which R2 is an amino acid residue selected from the group consisting of alanine, glycine, isoleucine, leucine, proline, methionine, phenylglycine, phenylalanine, valine, and asparagine;
or a pharmaceutically acceptable salt or solvate thereof.

4. The compound according to claim 1, in which, in Substituent group B, the substituent is a group represented by formula 2: 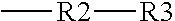
in which R3 is selected from the group consisting of methyl, ethyl, n-propyl, isopropyl, cyclopropyl, n-butyl, isobutyl, sec-butyl, tert-butyl, n-pentyl, isopentyl, sec-pentyl, tert-pentyl, neopentyl, 1-methylpentyl, n-hexyl, isohexyl, sec-hexyl, tert-hexyl, and neohexyl;
or a pharmaceutically acceptable salt or solvate thereof.

5. The compound according to claim 1, in which, in Substituent group B, the substituent is a group represented by formula 2: 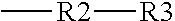
in which R3 is a straight-chain or branched C4 to C6 alkyl group;
or a pharmaceutically acceptable salt or solvate thereof.

6. The compound according to claim 1, in which the compound is represented by 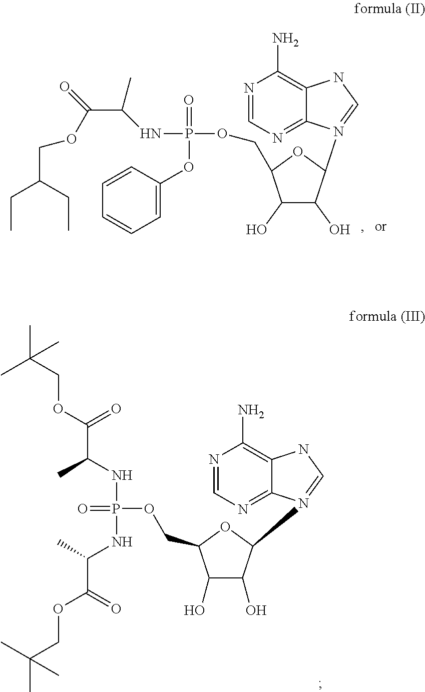
or a pharmaceutically acceptable salt or solvate thereof.

7. A pharmaceutical composition which comprises the compound according to claim 1, or a pharmaceutically acceptable salt or solvate thereof.

8. The pharmaceutical composition according to claim 7, in which the composition is for treating or preventing a state of decreased intracellular ATP levels.

9. The pharmaceutical composition according to claim 8, in which the state of decreased intracellular ATP levels is associated with an aging state, as well as a non-alcoholic fatty liver disease and/or hepatitis.

10. The pharmaceutical composition according to claim 8, in which the state of decreased intracellular ATP levels is a mitochondrial disease, a cancer, or a hypoxic condition due to insufficient blood flows, or a tissue regeneration area accompanied by inflammation.

11. The pharmaceutical composition according to claim 8, in which the state of decreased intracellular ATP levels is a neurodegenerative disease.

12. The pharmaceutical composition according to claim 11, in which the neurodegenerative disease is selected from the group consisting of Parkinson's disease, Alzheimer's disease, and ALS.
